(19) United States
(12) Patent Application Publication
(10) Pub. No.: US 2012/0184923 A1
(43) Pub. Date: Jul. 19, 2012

(54) ACTUATOR FOR SAFETY DEVICES FOR NEEDLES AND DISPOSABLE MEDICAL INSTRUMENTS

(76) Inventor: Mario Gargani, Firenze (IT)

(21) Appl. No.: 13/498,147

(22) PCT Filed: Sep. 24, 2010
(86) PCT No.: PCT/IB2010/002390

§ 371 Date: Mar. 25, 2012

(30) Foreign Application Priority Data
Sep. 25, 2009 (IT) FI2009A000206

(51) Int. Cl. A61M 5/00

(52) U.S. Cl. 604/263

(57) ABSTRACT

An actuator for safety devices (2) for needles of medical instruments of the type comprising two half-parts (3), which are connected together by a hinge line (4) and can be closed about a needle of a medical instrument to render it safe; the half-parts (3) each have an engagement portion (11) set at respective ends (3a) of the half-parts (3) to grip on the needle in a closing configuration of the half-parts (3); the actuator comprises a support (17) for supporting the half-parts (3) of the safety device (2), and a sliding slot (24) made in the support (17) in which the engagement portions (11) of the half-parts (3) can be slidably inserted; the sliding slot (24) has a width substantially equal to a width of the safety device (2) at the ends (3a) in the closing configuration of the half-parts (3) so as to bring them up to one another and push the half-parts (3) into the closing configuration.

### SECTOR OF THE ART

The subject of the present invention is an actuator for safety devices for needles and disposable medical instruments. Said invention finds application in rendering safe needles and other disposable medical instruments, such as for example syringes, catheters, winged needles, etc. In the framework of the present treatment reference is made to needles for syringes, without this implying any loss of generality.

### STATE OF THE ART

Generally, known safety devices are constituted by a cover that is applied on the needle to isolate it from the surrounding environment by enclosing it in a compartment that is not accessible to an operator.

For example, known safety devices envisage a cap with an opening in a position corresponding to one end so that it can be fitted by pressure on the needle. The cap is fitted on the needle through said opening, in the direction of development of the needle.

In a further embodiment, the cap has a longitudinal slit made along a wall of the cap itself. The needle is inserted into the cap through said slit, and is then constrained thereto, purely by way of example, by being slotted or engaged by pressure. In this way, the needle is covered with the cap and is no longer exposed, and is hence rendered safe. The needle can then be disposed of without any risks for the operator. In particular, the needle can be disposed of together with the syringe or separately.

In both of the configurations illustrated, the safety device is used by the operator who grips the safety device with one hand and holds with the other the syringe, which comprises the needle to be neutralized. Hence, the operator has to approach the needle with his hands in order to insert into the device and block it.

The use of known safety devices presents an important disadvantage. In particular, there is an evident risk of accidental injury for the operator during the step of rendering the needle safe, in so far as to carry out said step the operator has to approach the needle with his hand.

In this context, the technical task underlying the present invention is to propose an actuator for safety devices for needles of medical instruments that will overcome the drawbacks of the known art referred to above.

### PURPOSE OF THE INVENTION

In particular, the purpose of the present invention is to make available an actuator for safety devices for needles of medical instruments that will be able to guarantee a high level of safety for the operator while he is performing the operation of rendering the needle safe.

### SUMMARY OF THE INVENTION

The technical task referred to and the purpose specified are basically achieved by an actuator for safety devices for needles of medical instruments, comprising the technical characteristics set forth in one or more of the annexed claims.

### LIST OF THE DRAWINGS

Further characteristics and advantages of the present invention will emerge more clearly from the illustrative, and consequently non-limiting, description of a preferred but non-exclusive embodiment of an actuator for safety devices for needles of medical instruments, as illustrated in the annexed drawings, wherein:

FIG. 1 is a perspective view of an actuator for safety devices for needles of medical instruments according to the present invention;

FIG. 2 is a perspective view of the actuator for safety devices for needles of medical instruments of FIG. 1, inserted in which is a safety device according to a first mode of use;

FIG. 2a is a detail of the actuator for safety devices for needles of medical instruments of FIG. 2;

FIG. 3 is a perspective view of the actuator for safety devices for needles of medical instruments of FIG. 1, inserted in which is a safety device according to a second mode of use;

FIG. 4 is a perspective view of a tray comprising the actuator for safety devices for needles of medical instruments of FIG. 1;

FIG. 5 is a perspective view of a safety device that can be used together with the actuator for safety devices according to the invention;

FIG. 6 shows a second embodiment of an actuator according to the invention, in perspective view;

FIGS. 7 and 7a show respectively a top plan view and a plan view from beneath of the actuator of FIG. 6;

FIG. 8 shows a front view of the actuator of FIG. 7;

FIG. 9 shows a side view from the right of the actuator of FIG. 8;

FIG. 10 shows a side view from the left of the actuator of FIG. 8;

FIG. 11 shows the cross section A-A of the actuator of FIG. 8;

FIG. 12 shows a further embodiment of a safety device that can be used together with the actuator for safety devices according to the invention;

FIG. 13 shows a view from beneath of the safety device of FIG. 12;

FIG. 14 shows a front view of an actuator according to the invention provided with lever for activation of the safety device;

FIGS. 15 and 16 show, respectively, in side view from the left the positions of opening and of closing of the actuator of FIG. 14; and

FIG. 17 shows a containment tray with seat for housing an actuator according to the invention;

### DETAILED DESCRIPTION

With reference to the annexed figures, designated as a whole by 1 is an actuator for safety devices for needles of medical instruments in accordance with the present invention. For example, the medical instruments can be syringes, catheters, winged needles, etc. In what follows, reference will be made to a syringe, without this, however, implying any loss in generality. In particular, the actuator 1 can be used on a safety device 2 described hereinafter.

The safety device 2 comprises two half-parts 3 connected together by a hinge line 4, which are rotatably mobile about the hinge line 4 itself. Said half-parts 3 are substantially symmetrical. Said half-parts 3 can be closed irreversibly around the needle of a syringe to render said needle safe.

In particular, the half-parts 3 can assume an opening configuration, prior to use of the safety device 2, and a closing configuration, after use.

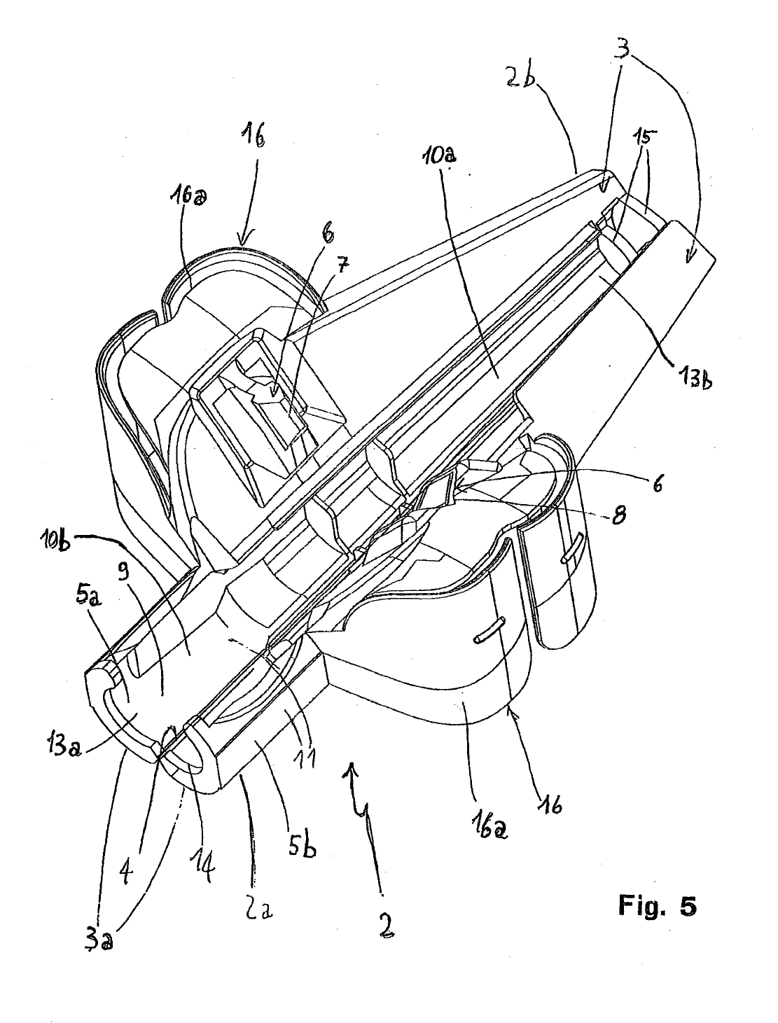

In particular, in the opening configuration, illustrated in FIG. 5, the two half-parts 3 are set at a distance apart and connected to one another only along the hinge line 4.

In the closing configuration, not illustrated, the two half-parts 3 face one another and are completely in contact.

The safety device 2 has an inner surface 5a and an opposite outer surface 5b.

On the inner surface 5a, irreversible closing means 6 are present. In particular, as illustrated in FIG. 5, preferably present on the inner surface 5a of one of the two half-parts 3 is a projecting element 7, which engages in a recess 8 set on the other half-part 3 in a position symmetrical with respect to the hinge line 4.

When the two half-parts 3 are in the closing configuration, and the internal surfaces 5a of the two half-parts 3 face one another, the projecting element 7 engages in the recess 8, and the safety device 2 can no longer be opened.

Each half-part 3 is concave, and moreover has a respective channel 9 that develops parallel to the hinge line 4. Each channel 9 defines half of a compartment for receiving a needle when the half-parts 3 are in the closing configuration.

Each half-part 3 moreover has a respective engagement portion 11 set in a corresponding end 3a of the half-part 3 itself to grip on the needle in the closing configuration. In said closing configuration, the two half-parts 3 turn about the hinge line 4, longitudinal and parallel to a longitudinal axis of prevalent development of the safety device 2. The two channels 9 of each half-part 3 hence fit together to form the compartment for receiving the needle.

The compartment is located centrally with respect to the safety device 2, along the longitudinal axis of prevalent development of the safety device 2.

The compartment moreover has a first cylindrical portion for containing the needle proper, and a second portion defined by the engagement portions of the half-parts 3 for containing a base of the needle.

A first end of the compartment, which corresponds to a first end 2a of the safety device 2, has a radial annular shoulder 14, which extends in elevation from the inner surface 5a, which defines, when the half-parts 3 are in the closing configuration, an edge for containing the needle. Said shoulder 14 defines at least in part the engagement portion 11 of each half-part 3. The ends 3a of both of the half-parts moreover coincide with the first end 2a of the safety device 2.

The second end of the compartment, which corresponds to a second end 2b of the safety device 2, has two tabs 15, which are also in elevation from the inner surface 5a and axially staggered with respect to one another, so that, when the safety device 2 is in the closing configuration, they are set on top of one another, thus closing the second end of the compartment completely. In this way, any possible leakage of liquid is prevented.

On the outer surface 5b, the safety device 2 has a grip 16. In particular, each half-part 3 has a corresponding portion 16a of a grip 16.

The safety device 2 is preferably made of a single piece of plastic material.

The actuator 1 for safety devices 2 for needles of medical instruments can be used together with the safety device 2 described above.

Said actuator 1 comprises a support 17 for supporting the half-parts 3 of the safety device 2. In particular, the support 17 develops in a longitudinal direction, and has a first end 17a and a second end 17b opposite to the first end 17a. In greater detail, the support has a plane of symmetry, coinciding with the longitudinal direction of development of the support 17.

Said support 17 comprises two side walls 18 facing one another for supporting laterally, each, a respective half-part 3. In particular, said side walls 18 develop in directions parallel to one another. In the embodiment illustrated in the attached figures, said side walls 18 are parallel to one another.

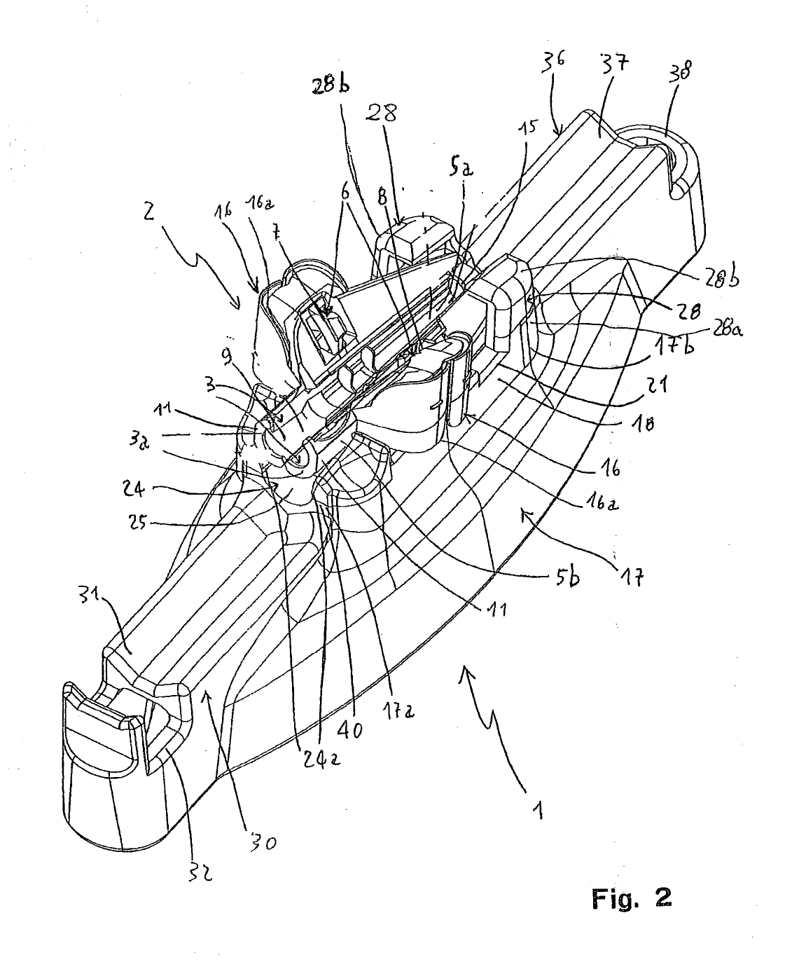

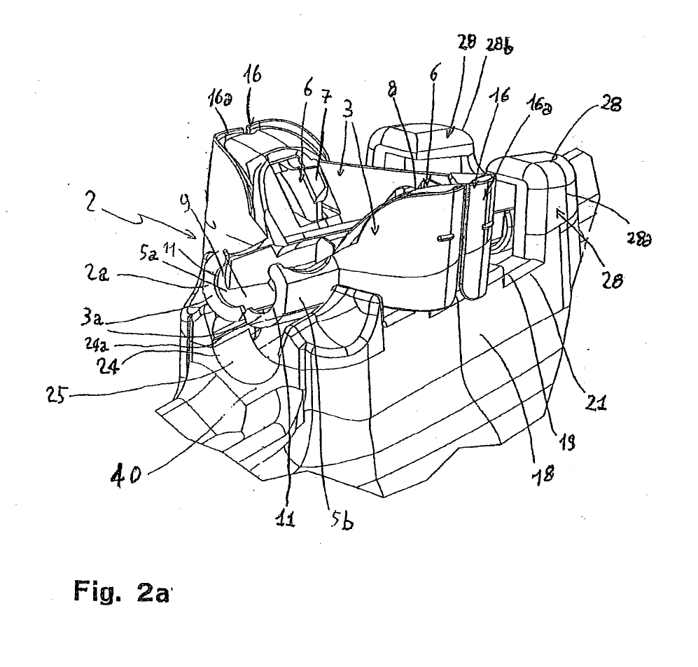

With reference to FIG. 2, each half-part 3 rests upon a respective side wall 18 in a position corresponding to its own portion 16a of grip 16. Advantageously, this enables the actuator 1 to stabilize the safety device 2 during use.

Each side wall 18 has a resting edge 19 shaped like a step to prevent any sliding of the safety device 2 in the longitudinal direction of development of the support 17. In other words, the resting edge 19 has a profile defined by a broken line. More precisely, the resting edge 19 has a profile defined by a plurality of segments 21 concatenated to one another, parallel, and lying in different planes.

The support 17 moreover has a cavity 22 inside it to enable the half-parts 3 to be introduced therein and to pass to the closing configuration. In particular, said cavity 22 is defined by the side walls 18.

According to what is illustrated, defined in the support 17 is a through opening oriented perpendicular to the longitudinal direction of development of the support 17. The opening is made between the side walls 18, and the aforementioned cavity 22 is set in a position corresponding to the opening.

In other words, the support 17 does not comprise a bottom wall in a position corresponding to the cavity 22.

Advantageously, this renders the actuator lighter, and facilitates sterilization thereof.

In one embodiment (not illustrated), the support 17 can have a bottom wall, set perpendicular to the side walls 18, to define, in combination with said side walls 18, the cavity 22.

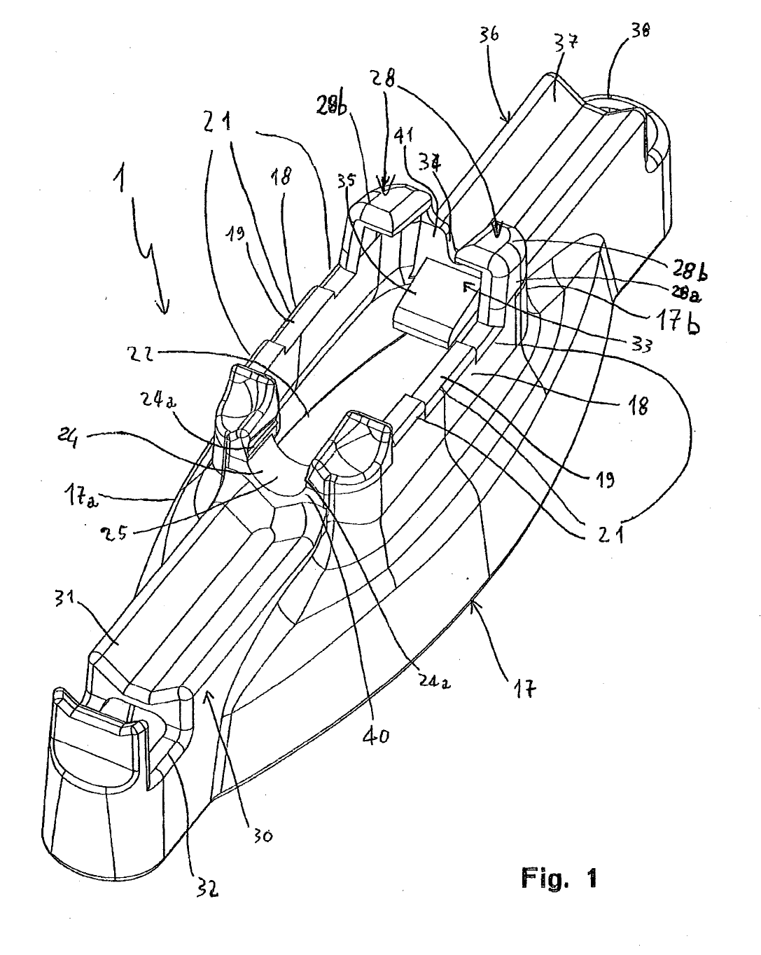

The actuator 1 has a sliding slot 24 made in the support 17 in which the engagement portions 11 of the half-parts 3 can be slidably inserted. In particular, the sliding slot 24 is set between the side walls 18 in a position corresponding to the first end 17a of the support 17. In the embodiment illustrated in FIG. 1, the sliding slot 24 is made on a transverse wall 40 perpendicular to the side walls 18 and set in a position corresponding to the first end 17a of the support 17.

Said sliding slot 24 has a width substantially equal to a width of the safety device 2 at the first end 2a of the safety device 2 itself when the half-parts 3 are in the closing configuration. Furthermore, the sliding slot 24 is concave, and has a bottom wall 25 of a shape at least in part complementary to the first end 2a of the safety device 2. In particular, in the embodiment described and illustrated, the first end 2a of the safety device 2 has a cylindrical shape when the half-parts 3 are in the closing configuration. Consequently, the bottom wall 25 is semi-cylindrical.

To render the needle of the syringe safe, the operator rests the safety device 2 on the actuator 1 by setting the engagement portion 11 in a position corresponding to the sliding slot 24.

Next, the operator pushes the syringe towards the inside of the sliding slot 24, inserting the needle between the two half-parts 3 of the safety device 2.

By pushing the syringe, the operator pushes the needle against the safety device 2 which drops into the sliding slot 24. Since the width of the sliding slot 24 is substantially equal to the width of the safety device 2 at the ends 3a of the half-parts 3 in their closing configuration, the engagement portions 11 are pushed by the sliding slot 24 itself against one another. In this way, the half-parts 3 reach the closing configuration. In this configuration, the engagement portions 11 are substantially in contact with the bottom wall 25 of the sliding slot 24.

The sliding slot 24 moreover has blocking projections 24a. Advantageously, this enables blocking of the engagement portions 11 when the half-parts 3 are in a closing configuration, and thus prevent re-opening of the safety device 2.

The actuator 1 further comprises at least one detent 28, set on at least one of the side walls 18 and can be engaged by the safety device 2. Said detent 28 is set substantially at the second end 17b of the support 17. Said detent 28 extends between the side walls 18 above the cavity 22. In use, when the safety device 2 is inserted into the actuator 1, the second end 2b of the safety device 2 is inserted under the detent.

Advantageously, this enables prevention of any misalignment between the safety device 2 and the support 17.

Preferably, the actuator 1 comprises two detents 28, each set on a respective side wall 18. In greater detail, each detent 28 comprises a first portion 28a, which extends from the side wall 18 on which it is made. Each detent 28 further comprises a second portion 28b for engaging on the safety device 2. Said second portions 28b of the respective detents 28 are orthogonal and develop approaching one another.

The actuator 1 comprises a housing 30 for a syringe; said housing 30 is connected to the support in a position corresponding to the sliding slot 24. In particular, said housing 30 extends in recession from the sliding slot 24, in the longitudinal direction of development of the support 17. The housing 30 has a concave resting surface 31 for the syringe, in such a way as to receive it and support it during the operation of rendering the needle safe. The housing 30 preferably has a recess 32 for facilitating insertion and extraction of the syringe. In particular, during the step of rendering the needle safe, the operator pushes the syringe towards the resting surface 31 in order to push the needle against the half-parts 3.

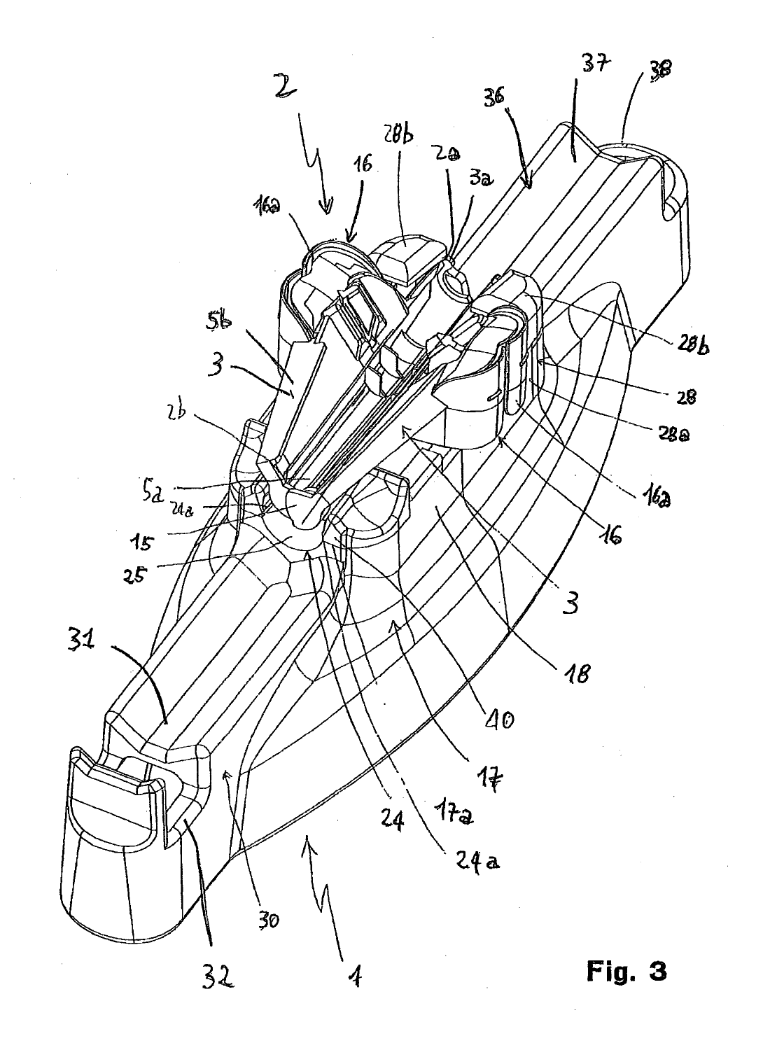

The safety device 2 can be set on the actuator 1 to be used according to a different mode (illustrated in FIG. 3) in order to render safe a medical instrument with geometrical characteristics different from those of the syringe, such as, purely by way of example, a cannula needle.

In this connection, the actuator 1 has a seat 33 for receiving the engagement portions 11, which is set between the side walls 18, at the second end 17b of the support 17. It should be noted that, in this case, the safety device 2 is fitted on the actuator 1 in a reversed position, as may be seen in FIG. 3. The receiving seat 33 is made on a further transverse wall 41 perpendicular to the side walls 18 and set at the second end 17b of the support 17.

The support 17 has a slit 34 facing the receiving seat 33. Said slit 34 has a width smaller than the width of the sliding slot 24 in such a way as to insert the cannula needle into the safety device 2.

The actuator 1 further comprises a support 35 for the safety device 2 in a position corresponding to the receiving seat 33, in such a way as to support the safety device 2 in the area of the engagement portion 11 when the half-parts 3 are in the closing configuration.

As illustrated in FIGS. 1 to 3, the actuator 1 comprises a further housing 36 for a cannula needle, connected to the support 17 in a position corresponding to the slit 34. In particular, said further housing 36 extends in the longitudinal direction of development of the support 17 moving away from the slit 34. The further housing 36 has a concave resting surface 37 of its own for the cannula needle in such a way as to receive it and support it during the operation of rendering the needle safe. The further housing 36 preferably has a recess 38 of its own for facilitating insertion and extraction of the cannula needle.

In order to render the cannula needle safe, the operator fits the safety device 2 on the actuator 1 by setting the first end 2a in a position corresponding to the second end 17b of the support in such a way as to set the engagement portions 11 of the half-parts 3 in a position corresponding to the receiving seat 33. Next, the operator brings the cannula needle closer to the further housing 36, inserting the needle between the two half-parts 3 of the safety device 2. Then the operator pushes the cannula needle towards the further resting surface 37 so that the needle in turn will push the half-parts 3 into the closing configuration. In this way, the needle is inserted into the compartment and neutralized. Advantageously, this enables cannula needles, or else other types of medical instruments of dimensions different from syringes, to be rendered safe.

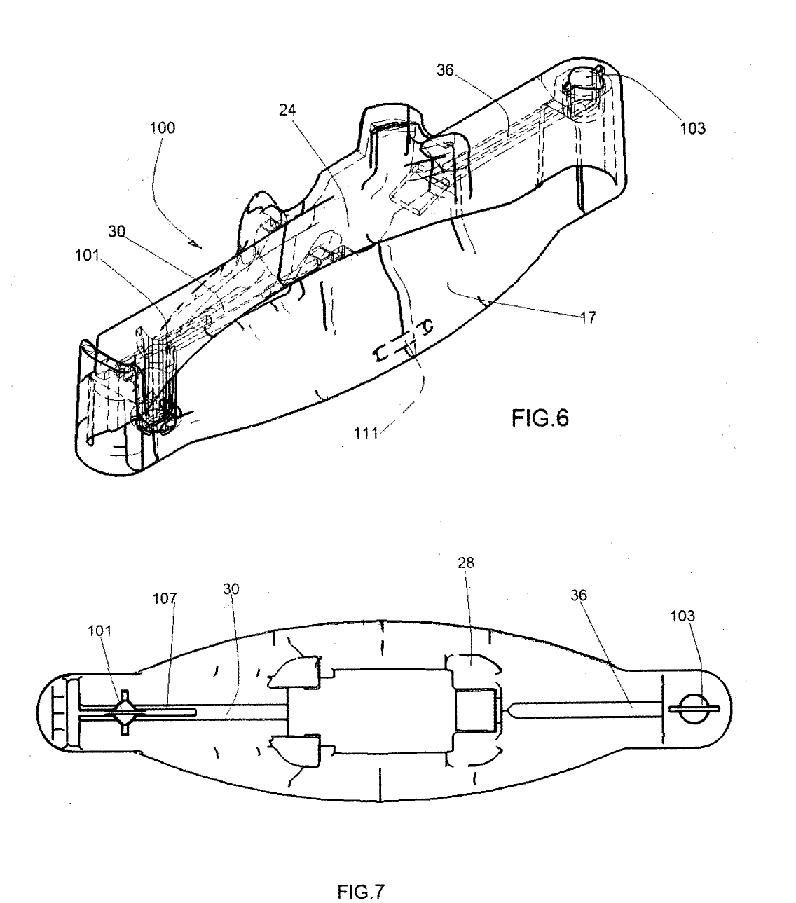

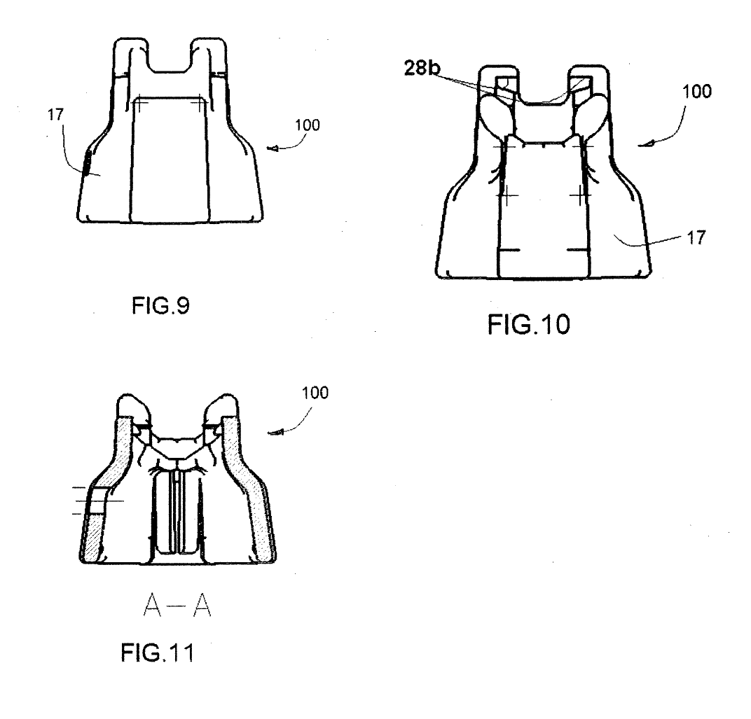

With reference to FIGS. 6-11, a second embodiment of an actuator 100 according to the invention is described, in which associated to corresponding parts are the same reference numbers already adopted for FIGS. 1-5.

The actuator 100 is provided with a hole 101 made along the resting seat 30 and is to house and withhold a needle cap for attachments of a Luer-Lok type. Advantageously, withholding of the needle cap is facilitated by a longitudinal cut 107 made along the resting seat 30, which bestows elasticity on the hole 101.

In use, the needle cover applied to the syringe (not illustrated because it is of a type in itself known) is inserted into the hole 101, and the syringe is extracted and used for the purpose envisaged.

After use of the syringe, the needle is once again inserted into the needle cover withheld by the hole 101, and the syringe is turned to release the needle from the Luer-Lok attachment.

The needle can thus be inserted into a device 2 so that it can be rendered safe and disposed of.

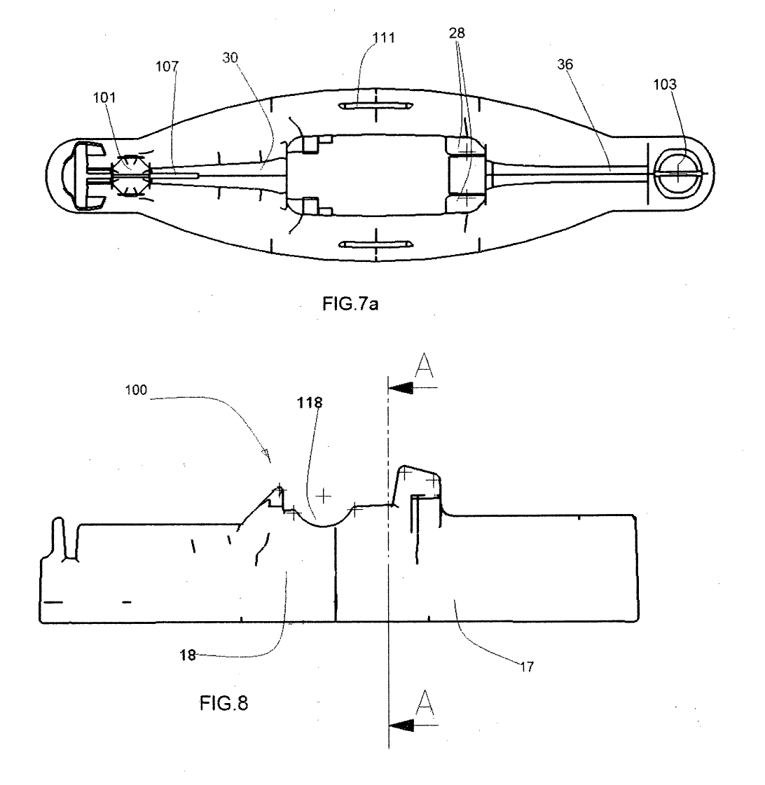

With reference once again to FIGS. 6 and 7, the actuator 100 is moreover provided with a hole 103 made along the resting seat 36 and is designed to house and withhold a needle cover for syringes for use in dentistry.

It should be noted that in the embodiment just described, the seat 33 is not provided with the cut 34 for housing instruments of the cannula needle type, which are also rested in the seat 30 with the device 2 positioned on the actuator, as illustrated in FIG. 2.

Preferably, as illustrated in FIG. 8, the walls 18 of the actuator 100 are moreover provided with openings 118, for example semicircular ones, set in positions corresponding to the points of application of the operator's fingers on the device 2 positioned in the cavity 24 of the actuator.

Advantageously, the cavities 118 enable a more convenient passage of the fingers at the moment of closing the device 2 and facilitate manoeuvring thereof.

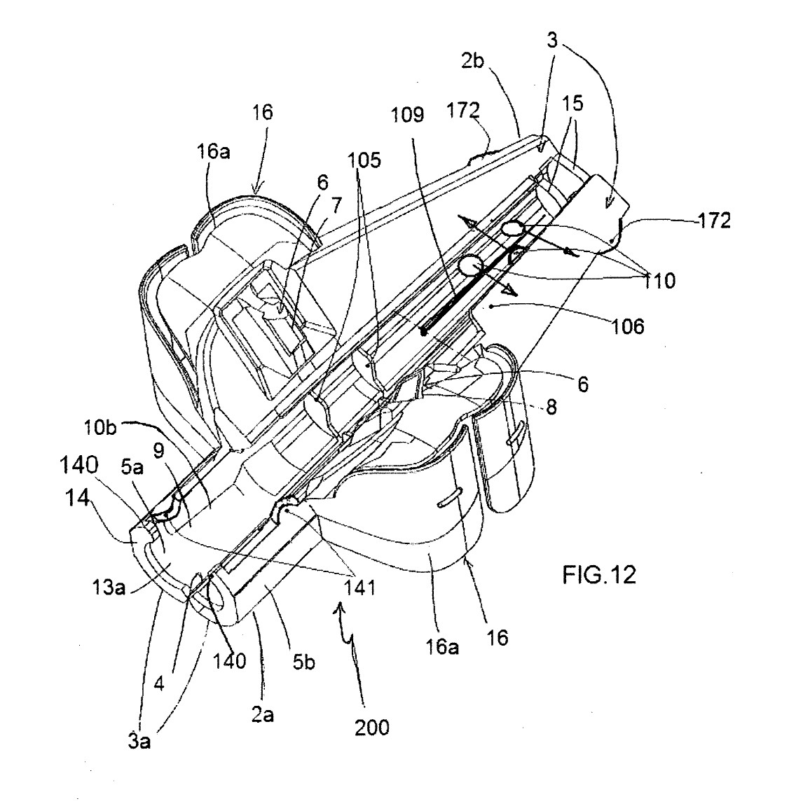

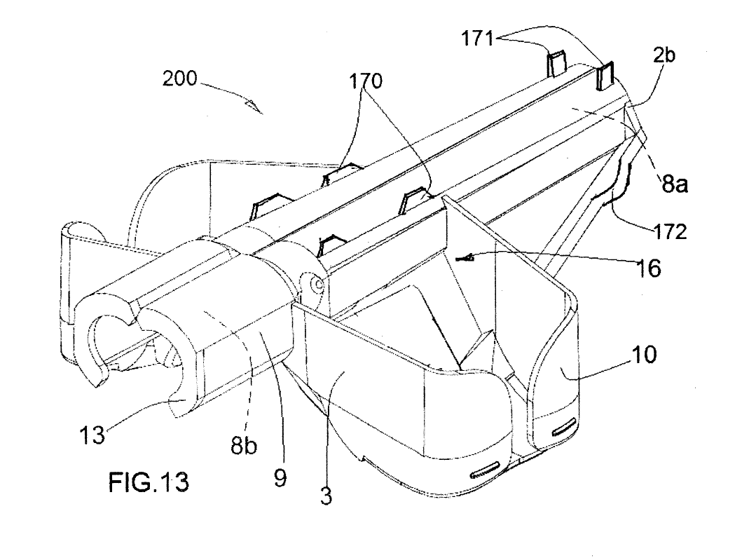

With reference to FIGS. 12 and 13, an embodiment of a safety device 200 is illustrated similar to the device 2 already described and in which parts in common are designated by the same reference numbers.

The device 200 is provided with crescents 172 set laterally to the superimposable top lamellae 106 of the half-parts 3 which have the function, as they bear upon the portions 28b of the detents 28, of preventing turning upwards of the advanced portion of the device 2 at the moment of resting of the medical instrument to be rendered safe.

Internally, the device 200 is moreover provided with transverse lamellae 105 that have the purpose of guiding positioning of the needle 109 within the device, whilst on the inner sides of the half-parts 3 one or more protrusions 110 are provided, which have the function of pressing on the needle, thus preventing re-use thereof.

In the example described, three protrusions 110 are provided, which are set staggered longitudinally, one on one side and the other two on the opposite side, and act on the needle 109, as indicated by the arrows in the figures, deforming it during closing of the device 200.

In the case of use with winged needles, the device 200 is moreover provided with respective prolongations 140 of the shoulders 14 of the engagement portions 11, which have the function of gripping the plastic lamellae of the wings once the device is closed, and of preventing exit thereof.

Furthermore, said engagement portions 11 can be provided with further protuberances 141 that serve to generate a friction on the lamellae of the wings at the moment of its insertion in the device in the open configuration, and prevent exit thereof prior to the closing manoeuvre.

Finally, illustrated in FIG. 13 is the bottom face of a safety device 200, which highlights a first distribution of feet 170 with the function both of providing a rest for the device and of preventing slipping of the operator's fingers when they are inserted in the grip 16.

Finally, present in the proximity of the front end 2b of the device is a second distribution of feet 171 with the function of keeping the portion 2b high once the device is housed in the actuator and limiting the risk that, during insertion of the needle in the device (in particular of a cannula needle), the latter projects from the device and prevents proper closing thereof.

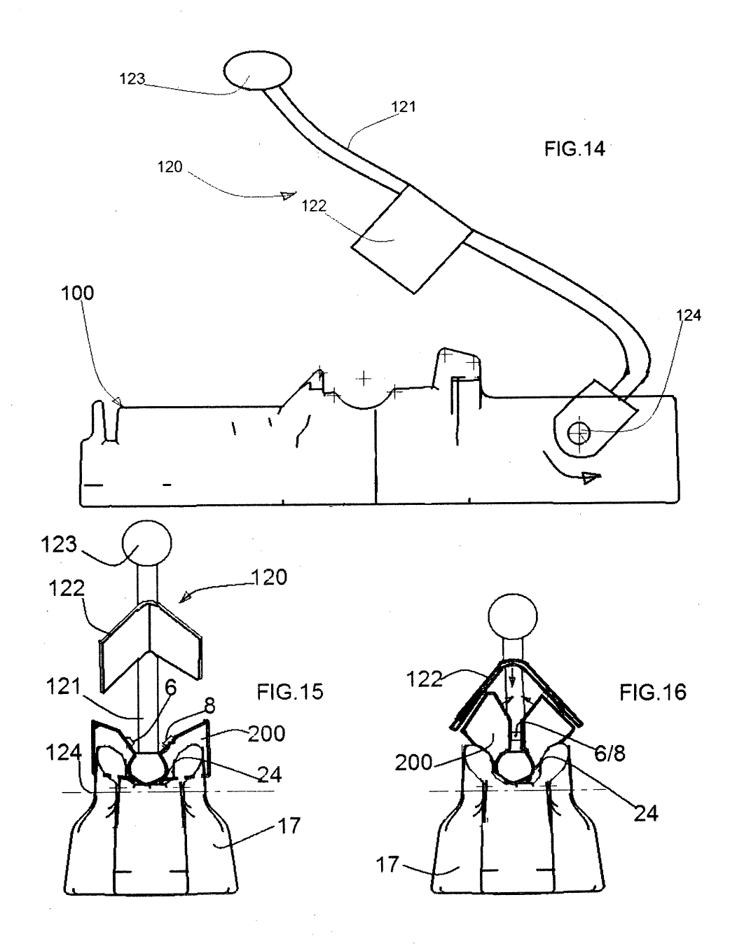

Illustrated schematically in FIGS. 14-16 is an embodiment of an actuator according to the invention, provided with a closing lever 120.

The lever 120 is normally in a high position with respect to the seat 24 of the actuator (FIGS. 14 and 15) for example thanks to a return spring (not illustrated) and can turn on a pin with transverse axis 124 to pass from the raised position to a lowered operating position in which a pressing hand 122 shaped like a V set upside down, fixed with respect to the rod 120, presses laterally on the two half-parts 3 of the device and closes them by causing the irreversible closing means 6-8 (FIG. 16) to intervene.

Advantageously, the use of a lever 120 prevents the operator from having to repeat manually the manoeuvre for closing the devices and further increases safety of handling of the medical instruments to be rendered safe.

It is understood that the shape of the lever may vary, for example assuming throughout its length a shaped like a V set upside down or other suitable shapes.

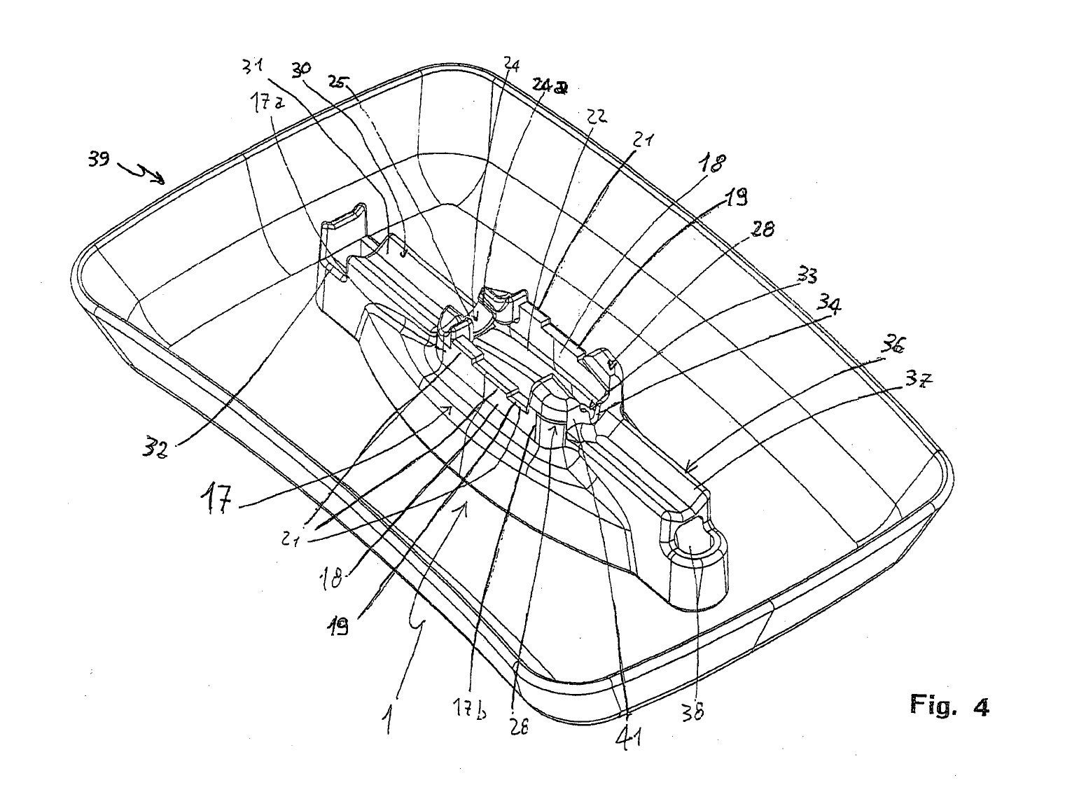

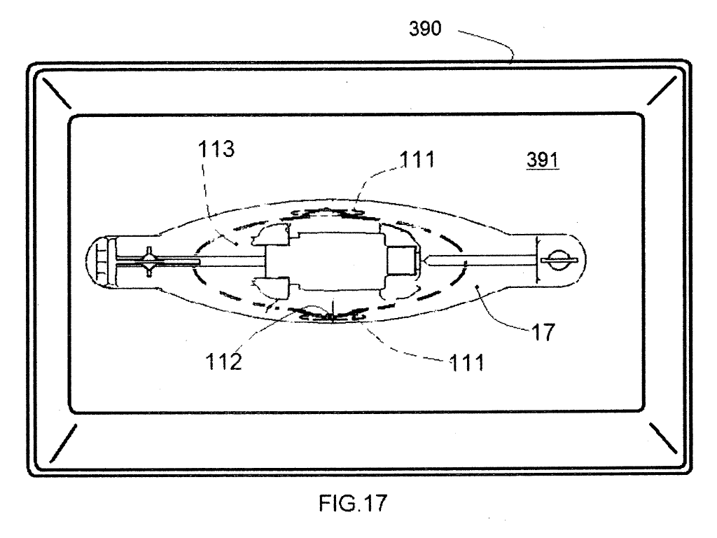

In accordance with the present invention and with particular reference to FIGS. 4 and 17, the actuator described above can be included in a tray 39/390 for medical use. Advantageously, this enables a greater convenience of use of the actuator 1/100.

Illustrated in FIG. 17 is a tray 390 provided with a dome 113 emerging from its bottom surface 391 and provided with lateral teeth 112 that are to engage by interference with corresponding shims 111 provided on the inner surface of the body 17 (more clearly visible in FIG. 7a).

Advantageously, with this solution, during use the actuator 100 remains stably but removably fixed in the tray 390.

The actuator 1/100 and the safety device 2/200, in the various embodiments illustrated can moreover be provided jointly in the form of a kit.

The present invention achieves the proposed purpose.

As has been seen, the operation of rendering the needle safe is performed by pushing the medical instrument so that the needle pushes the safety device on the support. Said action can be performed using the same hand with which the medical instrument is being held. Consequently, it is no longer necessary to bring the other hand up to the needle. In this way, the risk of accidental injections for the operator when rendering the needle safe is eliminated.

The actuator for safety devices for needles of medical instruments moreover affords an important advantage. Said actuator proves convenient to use in so far as it can be rested on a table or be inserted in a tray for medical use.

What is claimed is:

1. An actuator for a safety device (2) for needles and disposable medical instruments wherein said safety device comprise two half-parts (3), which are connected together by a hinge line (4) wherein said two half-parts are configured to close about a needle of a medical instrument to render said needle safe, said two half-parts (3) each having an engagement portion (11) set at respective ends (3a) of the two half-parts (3) configured to grip said needle in a closing configuration of said two half-parts (3) wherein said actuator comprises:
a support (17) configured to support said two half-parts (3) of a safety device (2), where said support comprises a sliding slot (24) in which each engagement portion (11) of the two half-parts (3) are configured to be slidably inserted;
said sliding slot (24) having a width substantially equal to a width of said safety device (2) at said respective ends (3a) in the closing configuration of said two half-parts (3) so as to bring said two half-parts up to one another and push said two half-parts (3) into the closing configuration.

2. The actuator according to claim 1, wherein said support (17) further comprises:
two facing side walls (18) configured to each laterally support a respective half-part and wherein said support (17) comprises a first end (17a) and a second end (17b) opposite to the first end (17a) and wherein said sliding slot (24) is located between said two facing side walls (18) at said first end (17a).

3. The actuator according to claim 2, wherein said support further comprises:
at least one detent (28), which is set on at least one of the two facing side walls (18) wherein said at least one detent is configured to be engaged by the safety device (2) to prevent any misalignment between the safety device (2) and the support (17) wherein said at least one detent (28) is located at the second end (17b) of the support (17).

4. The actuator according to claim 2, wherein said two facing side walls (18) each have a resting edge (19) comprising a step shape to prevent any sliding of the safety device (2) in a longitudinal direction of the support (17).

5. The actuator according to claim 1, further comprising:
a housing (30) for a medical instrument connected to the support (17) in a position proximal to said sliding slot (24).

6. The actuator according to claim 2, further comprising:
a seat (33) configured to receive each engagement portion (11) set between said two facing side walls (18) and at the second end (17b) of said support (17) wherein said support (17) comprises a slit (34) that faces the receiving seat (33) to enable insertion of said needle within the safety device (2).

7. The actuator according to claim 6, wherein said slit (34) has a width smaller than the width of said sliding slot (24).

8. The actuator according to claim 6 further comprising:
a further housing (36) for a medical instrument connected to the support (17) in a position proximal to said slit (34).

9. The actuator according to claim 2 wherein the two facing side walls (18) define between them a cavity (22) inside the support (17) to hold said two half-parts (3) therein to form the closing configuration.

10. The actuator according to claim 9, wherein the support (17) comprises a bottom wall that is perpendicular to the two facing side walls (18) wherein said bottom wall and said side walls (18) define the cavity (22).

11. The actuator according to claim 1, wherein said body said support (17) comprises a hole (101) made therein, wherein said hole is configured to house and removably withhold a needle cap of a hypodermic syringe or hypodermic needle.

12. The actuator according to claim 1, wherein said support (17) comprises a hole (103) that is configured to house and removably withhold a needle cover for syringes for use in dentistry.

13. The actuator according to claim 9, wherein the two facing side walls (18) of the actuator comprise openings (118) set in a position corresponding to points of application of an operator's fingers on the safety device (2) that is positioned in the cavity (22) of the actuator.

14. The actuator according to claim 1, further comprising a closing element configured to close a safety device (2, 200) set in the sliding slot (24) of the actuator.

15. The actuator according to claim 14, wherein said closing element comprises a lever or rod (120) that comprises a V-shaped element (122) that faces down with respect to the rod (120), wherein the rod (120) is configured to turn on a pin with transverse axis (124) to pass from a raised position to a lowered operating position, in which V-shaped element (122) presses laterally on said two half-parts (3) of the safety device.

16. The actuator according to claim 1, further comprising a tray for medical use for housing said actuator wherein said tray comprises a dome (113) emerging from a bottom surface (391) of said tray and provided with lateral teeth (112) that are configured to engage by interference with corresponding shims (111) provided on the inner surface of the support (17) of the actuator.

17. An actuator system comprising the actuator according to claim 1, and further comprising said safety device (2).
